(19) United States
(12) Patent Application Publication
(10) Pub. No.: US 2026/0022406 A1
(43) Pub. Date: Jan. 22, 2026

(54) Method for Improving Gas Production Efficiency of Anaerobic Fermentation System of Biomass Solid Waste

(71) Applicant: Northwest A&F University Shenzhen Research Inst, Shenzhen (CN)

(72) Inventors: Yiqing YAO, Shenzhen (CN); Tairan LIU, Shenzhen (CN); Hongyi LV, Shenzhen (CN)

(21) Appl. No.: 18/824,868

(22) Filed: Sep. 4, 2024

(30) Foreign Application Priority Data
Jul. 22, 2024 (CN) 202410980752.2

(51) Int. Cl. C12P 5/02; C12N 1/20; C12N 1/22

(52) U.S. Cl. CPC C12P 5/023; C12N 1/20; C12N 1/22

(57) ABSTRACT

A method for improving a gas production efficiency of an anaerobic fermentation system of a biomass solid waste is provided, belonging to the technical field of biomass energy and resource utilization. The method for improving a gas production efficiency of an anaerobic fermentation system of a biomass solid waste includes the following steps: mechanically stirring the biomass solid waste in a first-intensity mechanical stirring mode during a reaction start-up period; and mechanically stirring the biomass solid waste in a second-intensity mechanical stirring mode during a reaction peak period. The method is beneficial to hydrolysis and acidification in the start-up period of the anaerobic fermentation system, and does not inhibit activities of core microbial flora and key enzymes in the gas production stage.

### CROSS REFERENCE TO RELATED APPLICATION

This patent application claims the benefit and priority of Chinese Patent Application No. 202410980752.2 filed with the China National Intellectual Property Administration on Jul. 22, 2024, the disclosure of which is incorporated by reference herein in its entirety as part of the present application.

### TECHNICAL FIELD

A method for improving a gas production efficiency of an anaerobic fermentation system of biomass solid waste is provided, belonging to the technical field of biomass energy and resource utilization.

### BACKGROUND

The ever-increasing demand for energy by human beings has not only accelerated the rate of energy consumption, but also caused serious environmental problems. Anaerobic fermentation can convert various types of biomass waste into clean energy, enabling the high-value conversion of low-value resources while also mitigating the greenhouse effect and improving environmental problems.

Compared with traditional wet anaerobic fermentation, semi-dry and dry anaerobic fermentation have the advantages of less water demand, less biogas sludge produced after fermentation, and lower subsequent treatment costs. For existing research, mechanical stirring has predominantly been applied at a constant stirring intensity throughout the anaerobic fermentation process. This process aims to promote heat and mass transfer in semi-dry and dry anaerobic fermentation, thereby improving a gas production efficiency of the semi-dry and dry anaerobic fermentation.

However, there have been the following problems in the prior art:

In semi-dry and dry anaerobic fermentation, a constant stirring intensity throughout the whole process is adopted. If the stirring intensity is too low, it hinders the hydrolysis and acidification during the initial stage of the fermentation system, resulting in a significant extension of the reaction lag period and even startup failure.

Conversely, in semi-dry and dry anaerobic fermentation, if the constant stirring intensity throughout the whole process is too high, it may inhibit activities of core microbial flora and key enzymes in the gas production stage. This can result in a decrease in the gas production rate during the reaction peak period, ultimately leading to wasted energy.

Therefore, in the field of biomass energy and resource technology, it is highly important to find a mechanical stirring mode that is not only beneficial to the hydrolysis and acidification in the initial stage of the anaerobic fermentation system but also does not inhibit the activities of key enzymes in the gas production stage.

### SUMMARY

An objective of the present disclosure is to provide a method for improving a gas production efficiency of an anaerobic fermentation system of a biomass solid waste. The method is beneficial to hydrolysis and acidification in an initial stage of the anaerobic fermentation system, and does not inhibit activities of key enzymes in a gas production stage.

To achieve the above objective, the present disclosure provides the following technical solutions.

In a first aspect, the present disclosure provides a method for improving a gas production efficiency of an anaerobic fermentation system of a biomass solid waste, including the following steps:
mechanically stirring the biomass solid waste in a first-intensity mechanical stirring mode during a reaction start-up period; and
mechanically stirring the biomass solid waste in a second-intensity mechanical stirring mode during a reaction peak period.

In some embodiments, the first-intensity mechanical stirring mode is a high-intensity mechanical stirring mode; and
the second-intensity mechanical stirring mode is a low-intensity mechanical stirring mode.

In some embodiments, the high-intensity mechanical stirring mode is conducted 5 to 8 times per day, with each time lasting 21 min to 120 min; and

the low-intensity mechanical stirring mode is conducted 2 to 4 times per day, with each time lasting 5 min to 20 min.

In some embodiments, an inoculum in the anaerobic fermentation system is selected from the group consisting of a sludge from a sewage treatment plant, a fermentation feed liquid from a biogas project, and a sludge from a lotus pond.

In some embodiments, the sludge from the sewage treatment plant is an anaerobic microbial flora-rich sludge;
the fermentation feed liquid from the biogas project is an anaerobic microbial flora-rich fermentation feed liquid; and
the sludge from the lotus pond is the anaerobic microbial flora-rich sludge.

In some embodiments, the biomass solid waste is selected from the group consisting of crop straw, food waste, and livestock and poultry manure.

In some embodiments, the anaerobic fermentation system has a total solid content of 10% to 30%. For example, the anaerobic fermentation system may have a total solid content of 10%, 11%, 12%, 13%, 14%, 15%, 16%, 17%, 18%, 19%, 20%, 21%, 22%, 23%, 24%, 25%, 26%, 27%, 28%, 29%, or 30%.

In some embodiments, the anaerobic fermentation system is at 35° C. to 65° C. For example, the anaerobic fermentation system can be at 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C.

In some embodiments, the anaerobic fermentation system is selected from the group consisting of a semi-dry anaerobic fermentation system and a dry anaerobic fermentation system.

In a second aspect, the present disclosure provides a process for improving a methane production efficiency of an anaerobic fermentation system of a biomass solid waste, including implementing steps of the method.

In the present disclosure, semi-dry and dry anaerobic fermentation is a technology in which materials are decomposed by anaerobic fermentation microorganisms and then converted into clean products such as methane and biological organic fertilizer under less water conditions. Compared with wet anaerobic fermentation currently used in production, the dry anaerobic fermentation has the advantages of high material solid content, large raw material processing capacity, simple material feed and discharge, high volumetric gas production rate, low biogas slurry discharge, small reaction volume, low energy consumption, and easy management.

Compared with the conventional technology, the present disclosure has the following beneficial effects.

In the present disclosure, the biomass solid waste is mechanically stirred in a high-intensity mechanical stirring mode during the reaction start-up period, which can homogenize intermediate products in each spatial position of the semi-dry and dry anaerobic fermentation. This process improves a mass transfer diffusion coefficient, promotes heat and mass transfer, increases the activities of hydrolytic acidifying bacteria and hydrogenotrophic methanogens as well as enzymes related to the hydrolysis and acetic acid oxidation, accelerates substrate hydrolysis, and shortens the reaction lag period.

In the present disclosure, the biomass solid waste is mechanically stirred in a low-intensity mechanical stirring mode during the reaction peak period, which can prevent the mechanical stirring from inhibiting methanogenic archaea. Specifically, the biomass solid waste is mechanically stirred in the low-intensity mechanical stirring mode during the reaction peak period, thereby preventing mechanical stirring from reducing the gene abundance of enzymes related to DNA repair and substrate accessibility for methanogenic archaea and the activities of key enzymes catalyzing biochemical reactions in the methanogenic stage. Meanwhile, flow characteristics of the substrate at the reaction peak period have been significantly improved compared to those at the reaction start-up period. At this time, adjusting to the low-intensity mechanical stirring mode does not affect heat and mass transfer requirements of the anaerobic fermentation, and the low stirring intensity can reduce energy consumption.

Compared with semi-dry and dry anaerobic fermentations with constant stirring intensity or single stirring intensity, those with dynamically-adjusted mechanical stirring intensity of the present disclosure may effectively improve gas production efficiency, shorten the reaction cycle, and reduce energy loss, thereby reducing components and increasing output, and showing strong practicality and a wide range of applications. Correspondingly, compared with semi-dry and dry anaerobic fermentations with constant stirring intensity or single stirring intensity, those with dynamically-adjusted mechanical stirring intensity of the present disclosure may effectively improve methane production efficiency, shorten the reaction cycle, and reduce energy loss, thereby reducing components and increasing output, and showing strong practicality and a wide range of applications.

In addition, compared with semi-dry and dry anaerobic fermentations with other high-intensity mechanical stirring modes beyond the daily stirring times and each stirring time, those with dynamically-adjusted mechanical stirring intensity may effectively improve the methane production efficiency, shorten the reaction cycle, and reduce energy loss, thereby reducing components and increasing output. Similarly, compared with semi-dry and dry anaerobic fermentations with other low-intensity mechanical stirring modes other than the daily stirring times and each stirring time, those with dynamically-adjusted mechanical stirring intensity may effectively improve the methane production efficiency, shorten the reaction cycle, and reduce energy loss, thereby reducing components and increasing output.

### BRIEF DESCRIPTION OF THE DRAWINGS

To describe the technical solutions in the embodiments of the present disclosure more clearly, the following briefly introduces the accompanying drawings required for describing the examples. Apparently, the accompanying drawings in the following description show merely some embodiments of the present disclosure, and those of ordinary skill in the art may still derive other drawings from these accompanying drawings without creative efforts.

FIG. 1 shows cumulative methane yields of the semi-dry anaerobic fermentation of wheat straw under different stirring modes;

FIG. 2 shows cumulative methane yields of the dry anaerobic fermentation of wheat straw under different stirring modes;

FIG. 3 shows cumulative methane yields of the semi-dry anaerobic fermentation of cow dung under different stirring modes; and

FIG. 4 shows cumulative methane yields of the dry anaerobic fermentation of food waste under different stirring modes.

### DETAILED DESCRIPTION OF THE EMBODIMENTS

The technical solutions of the present disclosure will be described in detail below in conjunction with specific examples, but the protection scope of the present disclosure is not limited to the examples. The experimental methods described in the following examples are conventional methods unless otherwise specified; and the reagents and materials can be obtained from commercial sources unless otherwise specified.

### Example 1

This example provided semi-dry anaerobic fermentation of wheat straw under medium-temperature conditions, including the following steps,

Step 1, pretreatment of raw materials:

The wheat straw was collected as a test raw material (Yangling Demonstration Zone, Shaanxi Province), crushed through a 40-mesh sieve, and sealed and stored for later use. Relevant indicators of the wheat straw included: total solids (TS) content of 89.0%±0.02%, and a volatile solids (VS) content of 63.7%±1.3%.

The inoculum was sewage sludge (rich in anaerobic microbial flora). The preparation method of the inoculum included the followings. The sludge from a sewage treatment plant was mixed well with tap water in a mass ratio of 1:2, and then cultured at a constant temperature of 35° C. for 14 d to obtain the inoculum. Relevant indicators of the inoculum included a TS content of 10.0%±0.05%, and a VS content of 5.62%±0.01%.

Step 2, semi-dry anaerobic fermentation test:

A 100-L anaerobic fermentation reactor with an effective volume of 70 L was used, and the wheat straw was used as a substrate to allow fermentation at 35° C. In the test, there set up 2 experimental groups with constant stirring intensity and dynamic stirring intensity. The constant stirring intensity group (S1) had a stirring intensity of 4 times per day, with each time lasting 20 min; and the dynamic stirring intensity group (S2) had a stirring intensity of 5 times per day during the reaction start-up period, with each time lasting 21 min. When the daily methane yield exceeded 5 mL*g⁻¹*VS⁻¹, the stirring intensity was changed to 2 times per day, with each time lasting 10 min. The control group (CK) was not subjected to stirring.

The inoculum amount of sludge (based on VS) was 30%, and the total solid content of the anaerobic fermentation system was 12%. The tap water was added to the effective volume and mixed evenly, and nitrogen was introduced into the anaerobic fermentation reactor to replace the gas in an upper space of the fermentation reactor to form an anaerobic environment. Three replicates were set for each experimental group and control group. Gas was collected every 2 d and the methane volume was measured. The cumulative methane production of each group was monitored and the experiment was terminated only when gas production stopped.

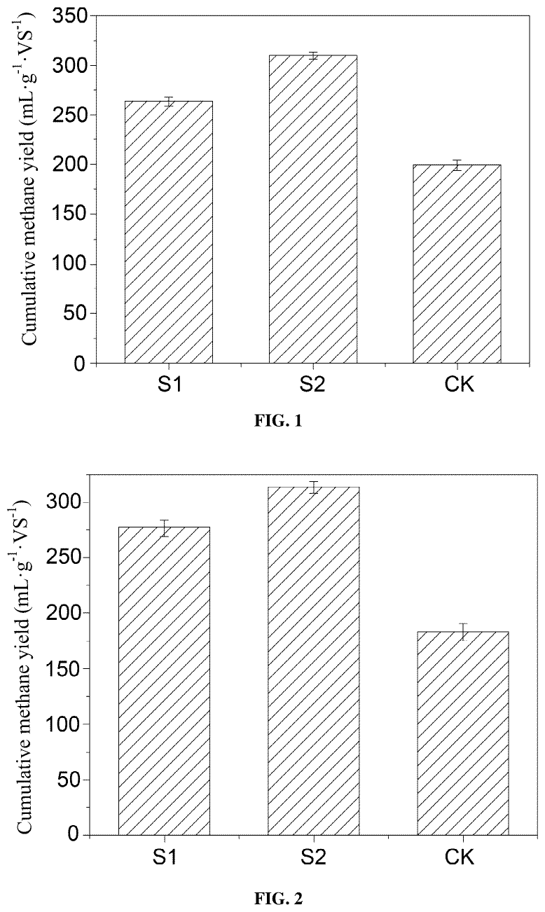

The results are shown in FIG. 1. As shown in FIG. 1, the average cumulative methane yields of the S1, S2, and CK groups were 263.8 mL*g⁻¹*VS⁻¹, 318.6 mL*g⁻¹*VS⁻¹, and 198.6 mL*g⁻¹*VS⁻¹, respectively; and the S2 group had the highest cumulative methane yield, which was 20.7% higher than that of the S1 group and 60.4% higher than that of the control group.

The above experimental results showed that compared with constant stirring intensity, the dynamically-adjusted stirring intensity increased the gas production rate of semi-dry anaerobic fermentation of wheat straw and reduced energy consumption, thereby effectively promoting the bacterial hydrolysis rate of wheat straw and the methane production capacity of methanogenic archaea during semi-dry anaerobic fermentation.

### Example 2

This example provided dry anaerobic fermentation of wheat straw under medium-temperature conditions, including the following steps.

Step 1, pretreatment of raw materials:

The wheat straw was collected as a test raw material (Yangling Demonstration Zone, Shaanxi Province), crushed through a 40-mesh sieve, and sealed and stored for later use. Relevant indicators of the wheat straw included a TS content of 89.0%±0.02%, and a VS content of 63.7%±1.3%.

The inoculum was sewage sludge (rich in anaerobic microbial flora). The preparation method of the inoculum included the following. The sludge from a sewage treatment plant was mixed well with tap water in a mass ratio of 1:2, and then cultured at a constant temperature of 35° C. for 14 d to obtain the inoculum. Relevant indicators of the inoculum included a TS content of 10.0%±0.05%, and a VS content of 5.62%±0.01%.

Step 2, dry anaerobic fermentation test:

A 100-L anaerobic fermentation reactor with an effective volume of 70 L was used, and the wheat straw was used as a substrate to allow fermentation at 35° C. In the test, there set up 2 experimental groups with constant stirring intensity and dynamic stirring intensity. The constant stirring intensity group (S1) had a stirring intensity of 4 times per day, with each time lasting 20 min; the dynamic stirring intensity group (S2) had a stirring intensity of 5 times per day during the reaction early period, with each time lasting 21 min. When the daily methane yield exceeded 5 mL*g⁻¹*VS⁻¹, the stirring intensity was changed to 2 times per day, with each time lasting 10 min. The control group (CK) was not subjected to stirring.

The inoculum amount of sludge (based on VS) was 30%, and the total solid content of the anaerobic fermentation system was 20%. The tap water was added to the effective volume and mixed evenly, and nitrogen was introduced into the anaerobic fermentation reactor to replace the gas in an upper space of the fermentation reactor to form an anaerobic environment. Three replicates were set for each experimental group and control group. Gas was collected every 2 d and the methane volume was measured. The cumulative methane production of each group was monitored and the experiment was terminated only when gas production stopped.

The results are shown in FIG. 2. As shown in FIG. 2, the average cumulative methane yields of the S1, S2, and CK groups were 276.5 mL*g⁻¹*VS⁻¹, 312.8 mL*g⁻¹*VS⁻¹, and 182.6 mL*g⁻¹*VS⁻¹, respectively; and the S2 group had the highest cumulative methane yield, which was 13.1% higher than that of the S1 group and 58.3% higher than that of the control group.

The above experimental results showed that compared with constant stirring intensity, the dynamically-adjusted stirring intensity increased the gas production rate of dry anaerobic fermentation of wheat straw and reduced energy consumption, thereby effectively promoting the bacterial hydrolysis rate of wheat straw and the methane production capacity of methanogenic archaea during dry anaerobic fermentation.

### Example 3

This example provided semi-dry anaerobic fermentation of cow dung under medium-temperature conditions, including the following steps:

Step 1, pretreatment of raw materials:

The fresh cow dung was collected as a test raw material (Yangling Demonstration Zone, Shaanxi Province). Relevant indicators of the cow dung included a TS content of 18.2%±0.06%, and a volatile solids (VS) content of 72.7%±1.3%.

The inoculum was sewage sludge (rich in anaerobic microbial flora). The preparation method of the inoculum included the followings. The sludge from a sewage treatment plant was mixed well with tap water in a mass ratio of 1:2, and then cultured at a constant temperature of 35° C. for 14 d to obtain the inoculum. Relevant indicators of the inoculum included a TS content of 10.0%±0.05%, and a VS of 5.62%±0.01%.

Step 2, semi-dry anaerobic fermentation test:

A 500-mL anaerobic fermentation reactor with an effective volume of 300 mL was used, and cow dung was used as a substrate to allow fermentation at 35° C. In the test, there set up 2 experimental groups with constant stirring intensity and dynamic stirring intensity. The constant stirring intensity group (S1) had a stirring intensity of 6 times per day, with each time lasting 25 min; and the dynamic stirring intensity group (S2) had a stirring intensity of 6 times per day during the reaction early period, with each time lasting 25 min. When the daily methane yield exceeded 5 mL*g⁻¹*VS⁻¹, the stirring intensity was changed to 4 times per day, with each time lasting 15 min. The control group (CK) was not subjected to stirring.

The inoculum amount of sludge (based on VS) was 30%, and the total solid content of the anaerobic fermentation system was 15%. The tap water was added to the effective volume and mixed evenly, and nitrogen was introduced into the anaerobic fermentation reactor to replace the gas in an upper space of the fermentation reactor to form an anaerobic environment. Three replicates were set for each experimental group and control group. Gas was collected every 2 d and the methane volume was measured. The cumulative methane production of each group was monitored and the experiment was terminated only when gas production stopped.

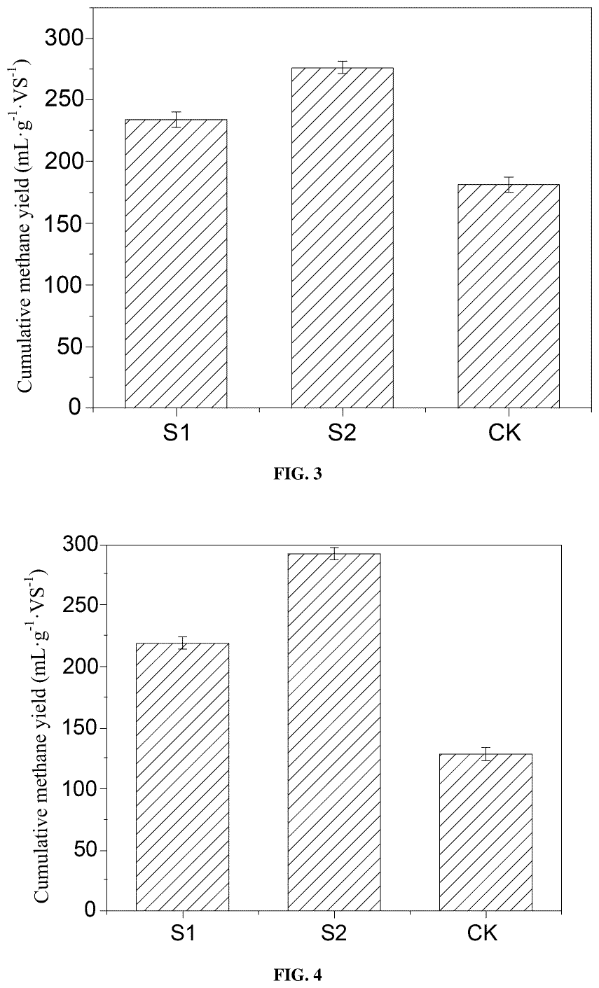

The results are shown in FIG. 3. As shown in FIG. 3, the average cumulative methane yields of the S1, S2, and CK groups were 232.8 mL*g⁻¹*VS⁻¹, 276.6 mL*g⁻¹*VS⁻¹, and 178.3 mL*g⁻¹*VS⁻¹, respectively; the S2 group had the highest cumulative methane yield, which was 19.2% higher than that of the S1 group and 55.1% higher than that of the control group.

The above experimental results showed that compared with constant stirring intensity, the dynamically-adjusted stirring intensity increased the gas production rate of semi-dry anaerobic fermentation of cow dung and reduced energy consumption, thereby effectively reducing the cost and time required for the degradation of cow dung, promoting the bacteria's ability to degrade substrates, enhancing the methane production potential of methanogenic archaea in the reactor, and increasing the net energy yield of anaerobic fermentation.

### Example 4

This example provided dry anaerobic fermentation of food waste under high-temperature conditions, including the following steps.

Step 1, pretreatment of raw materials:

The food waste was collected as a test raw material (student canteen of North Campus of Northwest Agriculture and Forestry University, Yangling Demonstration Zone, Shaanxi Province). Relevant indicators of the food waste included a TS content of 35.2%±0.06%, and a VS content of 92.6%±1.5%.

The inoculum was sewage sludge (rich in anaerobic microbial flora). The preparation method of the inoculum included the followings. The sludge from a sewage treatment plant was mixed well with tap water in a mass ratio of 1:2, and then cultured at a constant temperature of 35° C. for 14 d to obtain the inoculum. Relevant indicators of the inoculum included a TS content of 10.0%±0.05%, and a VS content of 5.62%±0.01%.

Step 2, dry anaerobic fermentation test:

A 500-mL anaerobic fermentation reactor with an effective volume of 300 mL was used, and food waste was used as a substrate to allow fermentation at 55° C. In the test, there set up 2 experimental groups with constant stirring intensity and dynamic stirring intensity. The constant stirring intensity group (S1) had a stirring intensity of 8 times per day, with each time lasting 30 min; and the dynamic stirring intensity group (S2) had a stirring intensity of 8 times per day during the reaction early period, with each time lasting 30 min. When the daily methane yield exceeded 5 mL*g⁻¹*VS⁻¹, the stirring intensity was changed to 4 times per day, with each time lasting 20 min. The control group (CK) was not subjected to stirring.

The inoculum amount of sludge (based on VS) was 30%, and the total solid content of the anaerobic fermentation system was 25%. The tap water was added to the effective volume and mixed evenly, and nitrogen was introduced into the anaerobic fermentation reactor to replace the gas in an upper space of the fermentation reactor to form an anaerobic environment. Three replicates were set for each experimental group and control group. Gas was collected every 2 d and the methane volume was measured. The cumulative methane production of each group was monitored and the experiment was terminated only when gas production stopped.

The results are shown in FIG. 4. As shown in FIG. 4, the average cumulative methane yields of the S1, S2, and CK groups were 220.7 mL*g⁻¹*VS⁻¹, 283.6 mL*g⁻¹*VS⁻¹, and 128.6 mL*g⁻¹*VS⁻¹, respectively; and the S2 group had the highest cumulative methane yield, which was 28.5% higher than that of the S1 group and 120.5% higher than that of the control group.

The above experimental results showed that compared with constant stirring intensity, the dynamically-adjusted stirring intensity increased the gas production rate of dry anaerobic fermentation of food waste and reduced energy consumption, thereby effectively promoting the activity of hydrolytic acidifying bacteria that degraded food waste and the methane production potential of methanogenic archaea, reducing the degradation cost and required time, and increasing the net energy yield of dry fermentation.

### Comparative Example 1

This comparative example provided dry anaerobic fermentation of food waste under high-temperature conditions, including the following steps.

Step 1, pretreatment of raw materials:

The food waste was collected as a test raw material (student canteen of North Campus of Northwest Agriculture and Forestry University, Yangling Demonstration Zone, Shaanxi Province). Relevant indicators of the food waste include a TScontent of 35.2%±0.06%, and a VS content of 92.6%±1.5%.

The inoculum was sewage sludge (rich in anaerobic microbial flora). The preparation method of the inoculum included the followings. The sludge from a sewage treatment plant was mixed well with tap water in a mass ratio of 1:2, and then cultured at a constant temperature of 35° C. for 14 d to obtain the inoculum. Relevant indicators of the inoculum included a TS content of 10.0%±0.05%, and a VS content of 5.62%±0.01%.

Step 2, dry anaerobic fermentation test:

A 500-mL anaerobic fermentation reactor with an effective volume of 300 mL was used, and food waste was used as a substrate to allow fermentation at 55° C. In the test, there set up 2 experimental groups with constant stirring intensity and dynamic stirring intensity. The constant stirring intensity group (S1) had a stirring intensity of 8 times per day, with each time lasting 30 min; and the dynamic stirring intensity group (S2) had a stirring intensity of 10 times per day during the reaction early period, with each time lasting 18 min. When the daily methane yield exceeded 5 mL*g⁻¹*VS⁻¹, the stirring intensity was changed to 4 times per day, with each time lasting 20 min. The control group (CK) was not subjected to stirring.

The inoculum amount of sludge (based on VS) was 30%, and the total solid content of the anaerobic fermentation system was 25%. The tap water was added to the effective volume and mixed evenly, and nitrogen was introduced into the anaerobic fermentation reactor to replace the gas in an upper space of the fermentation reactor to form an anaerobic environment. Three replicates were set for each experimental group and control group. Gas was collected every 2 d and a methane volume was measured. The cumulative methane production of each group was monitored and the experiment was terminated only when gas production stopped.

The results showed that the average cumulative methane yields of the S1, S2, and CK groups were 220.7 mL*g⁻¹*VS⁻¹, 210.8 mL*g⁻¹*VS⁻¹, and 128.6 mL*g⁻¹*VS⁻¹, respectively; and the S1 group had the highest cumulative methane yield, which was 4.69% higher than that of the S2 group and 71.2% higher than that of the control group.

### Comparative Example 2

This comparative example provided dry anaerobic fermentation of food waste under high-temperature conditions, including the following steps:

Step 1, pretreatment of raw materials:

The food waste was collected as a test raw material (student canteen of North Campus of Northwest Agriculture and Forestry University, Yangling Demonstration Zone, Shaanxi Province). Relevant indicators of the food waste included a TS content of 35.2%±0.06%, and aVS content of 92.6%±1.5%.

The inoculum was sewage sludge (rich in anaerobic microbial flora). The preparation method of the inoculum included the followings. The sludge from a sewage treatment plant was mixed well with tap water in a mass ratio of 1:2, and then cultured at a constant temperature of 35° C. for 14 d to obtain the inoculum. Relevant indicators of the inoculum included a TS content of 10.0%+0.05%, and a VS content of 5.62%=0.01%.

Step 2, dry anaerobic fermentation test:

A 500-mL anaerobic fermentation reactor with an effective volume of 300 mL was used, and food waste was used as a substrate to allow fermentation at 55° C. In the test, there set up 2 experimental groups with constant stirring intensity and dynamic stirring intensity. The constant stirring intensity group (S1) had a stirring intensity of 8 times per day, with each time lasting 30 min; and the dynamic stirring intensity group (S2) had a stirring intensity of 8 times per day during the reaction early period, with each time lasting 30 min. When the daily methane yield exceeded 5 mL*g⁻¹*VS⁻¹, the stirring intensity was changed to 5 times per day, with each time lasting 4 min. The control group (CK) was not subjected to stirring.

The inoculum amount of sludge (based on VS) was 30%, and the total solid content of the anaerobic fermentation system was 25%. The tap water was added to the effective volume and mixed evenly, and nitrogen was introduced into the anaerobic fermentation reactor to replace the gas in an upper space of the fermentation reactor to form an anaerobic environment. Three replicates were set for each experimental group and control group. Gas was collected every 2 d and a methane volume was measured. The cumulative methane production of each group was monitored and the experiment was terminated only when gas production stopped.

The results showed that the average cumulative methane yields of the S1, S2, and CK groups were 220.7 mL*g⁻¹*VS⁻¹, 198.6 mL*g⁻¹*VS⁻¹, and 128.6 mL*g⁻¹*VS⁻¹, respectively; and the S1 group had the highest cumulative methane yield, which was 11.1% higher than that of the S2 group and 71.2% higher than that of the control group.

The above test results show that, compared with semi-dry and dry anaerobic fermentations with other high-intensity mechanical stirring modes beyond the daily stirring times and each stirring time, those with dynamically-adjusted mechanical stirring intensity can effectively improve the methane production efficiency, shorten the reaction cycle, and reduce energy loss, thereby reducing components and increasing output. Similarly, compared with semi-dry and dry anaerobic fermentations with other low-intensity mechanical stirring modes other than the daily stirring times and each stirring time, those with dynamically-adjusted mechanical stirring intensity can effectively improve the methane production efficiency, shorten the reaction cycle, promote substrate degradation, and enhance the reactor's methane production potential, thereby reducing components and increasing output.

It is to be understood that the present disclosure is not limited to the particular methodology, protocols, and materials described as these may vary. It is also to be understood that the terminology used herein is for the purpose of describing particular embodiments only, and is not intended to limit the scope of the present disclosure which will be limited only by the appended claims.

Those skilled in the art will also recognize, or be able to ascertain using no more than routine experimentation, many equivalents to the specific embodiments of the present disclosure described herein. Such equivalents are also intended to be encompassed by the following claims. It should be noted that, the embodiments in the description are described in a progressive manner. Each embodiment focuses on the difference from other embodiments, and the same and similar parts between the embodiments may refer to each other.

Although preferred examples of the present application have been described, persons skilled in the art can make changes and modifications to these embodiments once they learn the basic inventive concept. Therefore, the appended claims are intended to be construed as covering the preferred examples and all changes and modifications falling within the scope of the embodiments of the present disclosure.

It should be noted that terms “including”, “comprising” or any other variants thereof are intended to cover non-exclusive inclusion, so that a process, method, article, or terminal device including a series of elements includes not only those elements but also other elements not explicitly listed, or elements inherent to such a process, method, article, or terminal device. Without more restrictions, the elements defined by the sentence “including a . . . ” do not exclude the existence of other identical elements in the process, method, article, or terminal device including the elements.

The technical solutions provided by the examples of the present disclosure are described in detail above, and the principles and implementations of the examples of the present disclosure are described herein by using specific examples. The description of the above examples is provided merely to help understand the principles of the examples of the present disclosure. In addition, those of ordinary skill in the art can make changes to the specific embodiments and application scope according to the examples of the present disclosure. In conclusion, the content of this specification should not be construed as limiting the present disclosure.

What is claimed is:

1. A method for improving a gas production efficiency of an anaerobic fermentation system of biomass solid waste, comprising the following steps:
mechanically stirring the biomass solid waste in a first-intensity mechanical stirring mode during a reaction start-up period; and
mechanically stirring the biomass solid waste in a second-intensity mechanical stirring mode during a reaction peak period.

2. The method according to claim 1, wherein
the first-intensity mechanical stirring mode is a high-intensity mechanical stirring mode; and
the second-intensity mechanical stirring mode is a low-intensity mechanical stirring mode.

3. The method according to claim 2, wherein
the high-intensity mechanical stirring mode is conducted 5 to 8 times per day, with each time lasting 21 min to 120 min; and
the low-intensity mechanical stirring mode is conducted 2 to 4 times per day, with each time lasting 5 min to 20 min.

4. The method according to claim 1, wherein
an inoculum in the anaerobic fermentation system is selected from the group consisting of a sludge from a sewage treatment plant, a fermentation feed liquid from a biogas project, and a sludge from a lotus pond.

5. The method according to claim 4, wherein
the sludge from the sewage treatment plant is an anaerobic microbial flora-rich sludge;
the fermentation feed liquid from the biogas project is an anaerobic microbial flora-rich fermentation feed liquid; and
the sludge from the lotus pond is the anaerobic microbial flora-rich sludge.

6. The method according to claim 1, wherein
the biomass solid waste is selected from the group consisting of crop straw, food waste, and livestock and poultry manure.

7. The method according to claim 1, wherein the anaerobic fermentation system has a total solid content of 10% to 30%.

8. The method according to claim 1, wherein the anaerobic fermentation system is at 35° C. to 65° C.

9. The method according to claim 1, wherein the anaerobic fermentation system is selected from the group consisting of a semi-dry anaerobic fermentation system and a dry anaerobic fermentation system.

10. A method for improving a methane production efficiency of an anaerobic fermentation system of a biomass solid waste, comprising conducting the method according to claim 1.

11. The method according to claim 10, wherein
the first-intensity mechanical stirring mode is a high-intensity mechanical stirring mode; and
the second-intensity mechanical stirring mode is a low-intensity mechanical stirring mode.

12. The method according to claim 11, wherein
the high-intensity mechanical stirring mode is conducted 5 to 8 times per day, with each time lasting 21 min to 120 min; and
the low-intensity mechanical stirring mode is conducted 2 to 4 times per day, with each time lasting 5 min to 20 min.

13. The method according to claim 10, wherein
an inoculum in the anaerobic fermentation system is selected from the group consisting of a sludge from a sewage treatment plant, a fermentation feed liquid from a biogas project, and a sludge from a lotus pond.

14. The method according to claim 13, wherein
the sludge from the sewage treatment plant is an anaerobic microbial flora-rich sludge;
the fermentation feed liquid from the biogas project is an anaerobic microbial flora-rich fermentation feed liquid; and
the sludge from the lotus pond is the anaerobic microbial flora-rich sludge.

15. The method according to claim 10, wherein
the biomass solid waste is selected from the group consisting of crop straw, food waste, and livestock and poultry manure.

16. The method according to claim 10, wherein the anaerobic fermentation system has a total solid content of 10% to 30%.

17. The method according to claim 10, wherein the anaerobic fermentation system is at 35° C. to 65° C.

18. The method according to claim 10, wherein the anaerobic fermentation system is selected from the group consisting of a semi-dry anaerobic fermentation system and a dry anaerobic fermentation system.
